(19) United States
(12) Patent Application Publication
(10) Pub. No.: US 2021/0361277 A1
(43) Pub. Date: Nov. 25, 2021

(54) SUTURE ANCHOR

(71) Applicant: IMPLAMENT PTY LTD, Currambine (AU)

(72) Inventor: Daniel Johannes PIENAAR, Currambine (AU)

(21) Appl. No.: 17/055,983

(22) PCT Filed: May 16, 2019
(86) PCT No.: PCT/AU2019/050466

§ 371 Date: Nov. 16, 2020

(30) Foreign Application Priority Data
May 17, 2018 (AU) 2018901728

(51) Int. Cl. A61B 17/04

(52) U.S. Cl. CPC A61B 17/0401; A61B 2017/0414; A61B 2017/0464; A61B 2017/0459; A61B 2017/0456

(57) ABSTRACT

Provided is a suture anchor 10 for securing soft tissue 12 to bone 6. The suture anchor 10 comprises a strip 14 having first and second end portions 16 and 18 and a central portion 20 therebetween. Each end portion 16 and 18 defines an inner and an outer aperture 22 and 24, said central portion 20 defining a central aperture 21 and transverse lines of weakness 26 longitudinally on either side of said central aperture 21. A suture 28 is operatively threadable through the strip 14 such that the end portions 16 and 18 fold onto each other via the lines of weakness 26 to complementarily define a clasp portion 30 with the central portion 20 defining an expansion portion 32. In use, tensioning of the suture 28 urges the expansion portion 32 into an expanded position and clasps said suture 28 to the clasp portion 30.

### TECHNICAL FIELD

This invention relates to a suture anchor for securing soft tissue to bone, and an associated method for securing soft tissue to bone.

### BACKGROUND ART

The following discussion of the background art is intended to facilitate an understanding of the present invention only. The discussion is not an acknowledgement or admission that any of the material referred to is or was part of the common general knowledge as at the priority date of the application.

In the field of medicine, it is an increasingly common problem for tendons and other soft, connective tissues to tear or to detach from associated bone. One such type of tear or detachment is a “rotator cuff” tear in the shoulder, wherein the supraspinatus tendon separates from the humerus, causing pain and loss of ability to elevate and externally rotate the arm. Complete separation can occur if the shoulder is subjected to gross trauma, but typically, the tear begins as a small lesion, especially in older patients. Similarly, other injuries can occur in various parts of the body.

When soft tissue tears away from bone, reattachment becomes necessary. Various devices, including sutures alone, screws, staples, wedges, and plugs have been used in the prior art to secure soft tissue to bone.

Suture anchors are generally comprised of the following basic parts: an anchor, an eyelet and a suture. The anchor is the portion that can be inserted into the bone. The anchor may conventionally be a screw or plug mechanism and made of metal or biodegradable material that can dissolve in the body over time. The eyelet is a hole or a loop in the anchor through which the suture passes thereby linking the anchor to the suture. Conventional suture anchors secure a suture by securing the suture by an internal screw plug, capturing the suture via friction between a bone tunnel and an implant or capturing the suture through one-way slip knots. Alternatively, other conventional suture anchors feature eyelets placed so that the suture is secured by means of a knot within the anchor itself.

Problems can arise if the anchor detaches from bone, or if a knot or eyelet arrangements for securing the suture fails, thereby allowing the suture to become detached from the anchor.

The current invention was conceived with these shortcomings in mind and proposes to ameliorate the shortcomings in the art by providing a suture anchor with improved attachment performance for accommodating one or more suture threads, which is simple in structure and easy and inexpensive to manufacture and use.

### SUMMARY OF THE INVENTION

According to a first aspect of the invention there is provided a suture anchor for securing soft tissue to bone, said suture anchor comprising:

a strip having first and second end portions and a central portion therebetween, each end portion defining an inner and an outer aperture, said central portion defining a central aperture and transverse lines of weakness longitudinally on either side of said central aperture;

wherein a suture is operatively threadable through the strip such that the end portions fold onto each other via the lines of weakness to complementarily define a clasp portion with the central portion defining an expansion portion; and wherein, in use, tensioning of said suture urges the expansion portion into an expanded position and clasps said suture to the clasp portion.

Typically, the suture is sequentially threaded through the strip as follows:
i) through the central aperture;
ii) via the inner aperture in the first end portion;
iii) via the outer aperture in the first end portion;
iv) via the outer aperture in the second end portion;
v) via the inner aperture in the second end portion;
vi) returning to the outer aperture in the first end portion;
vii) via the inner aperture in the first end portion; and
viii) exiting the strip via the central aperture.

Typically, the strip is manufactured from medical-grade polymer material.

In an embodiment, the clasp portion defines a rough surface texture to facilitate in operatively clasping the suture.

Typically, the strip includes further lines of weakness to facilitate in the expansion portion urging into the expanded position.

In an embodiment, the strip defines an additional line of weakness transversely across the central aperture.

In an embodiment, the strip defines two additional lines of weakness to separate the clasp portion from the expansion portion.

In an embodiment, the suture anchor includes a locking suture operatively threaded through the strip and having a detent configured to lock the expanded portion into the expanded position.

In an embodiment, the detent of the locking suture comprises a bulge sized, shaped and dimensioned for interference fit through an aperture of the strip.

In an embodiment, the strip includes an end portion guide configured to guide the end portions once folded onto each other, the end portion guide configured to allow sliding of such end portions longitudinally relative to each other.

In an embodiment, the strip includes a locking mechanism configured to lock the end portions together once folded onto each other.

In an embodiment, the locking mechanism is configured to complementarily engage the end portions to facilitate the clasp portion clasping the suture.

In an embodiment, the locking mechanism includes a cannulated screw configured to facilitate in urging and/securing the expansion portion into the expanded position.

In an embodiment, the first and/or second end portions define one or more opening leading into the inner and/or outer apertures for a suture to operatively pass through, said opening configured to allow suture entry into said inner and/or outer aperture, while substantially impeding suture exit therefrom.

According to a second aspect of the invention there is provided a method for securing soft tissue to bone, said method comprising the steps of:

providing a suture anchor comprising a strip having first and second end portions and a central portion therebetween, each end portion defining an inner and an outer aperture, said central portion defining a central aperture and transverse lines of weakness longitudinally on either side of said central aperture;

threading a suture through the strip such that the end portions fold onto each other via the lines of weakness to complementarily define a clasp portion with the central portion defining an expansion portion, said suture attachable to soft tissue;

inserting the folded strip clasp portion-first into a bone channel; and

tensioning the suture to urge the expansion portion into an expanded position to secure said strip inside said bone channel and to clasp said suture to the clasp portion.

Typically, the method includes the step of attaching the suture to soft tissue to be secured proximate the bone channel.

Typically, the step of threading the suture includes sequentially threading the suture:
i) through the central aperture;
ii) via the inner aperture in the first end portion;
iii) via the outer aperture in the first end portion;
iv) via the outer aperture in the second end portion;
v) via the inner aperture in the second end portion;
vi) returning to the outer aperture in the first end portion;
vii) via the inner aperture in the first end portion; and
viii) exiting the strip via the central aperture.

### BRIEF DESCRIPTION OF THE DRAWINGS

The description will be made with reference to the accompanying drawings in which:

FIGS. 1A and 1B are diagrammatic side-view and side-sectional view representations of the suture anchor strip, in accordance with an aspect of the present invention;

FIGS. 2A and 2B are diagrammatic perspective-view representations of the suture anchor strip of FIG. 1m, when folded on itself and without a suture;

FIG. 3 is a diagrammatic perspective-view representation of the suture anchor of FIG. 1 threaded with a suture prior to use;

FIG. 4 is a diagrammatic side-sectional view representation of the suture anchor of FIG. 1 wherein the suture is undergoing tensioning showing the expansion portion being urged into the expanded position;

FIG. 5 is a diagrammatic side-sectional view representation of the suture anchor of FIG. 1 with the expansion portion urged into the expanded position;

FIG. 6 is a diagrammatic side-sectional view representation of the suture anchor of FIG. 1 when inserted into a bone channel;

FIG. 7 is a diagrammatic side-sectional view representation of the suture anchor of FIG. 1 when inserted into a bone channel wherein the suture is tensioned showing the expansion portion urged into the expanded position;

FIG. 8 is a diagrammatic perspective-view representation of a further embodiment of the suture anchor, in accordance with an aspect of the invention, threaded with a suture prior to use;

FIG. 9 is a diagrammatic perspective-view representation of the suture anchor of FIG. 1 with a locking suture;

FIG. 10 is a diagrammatic perspective-view representation of the clasp portion of the suture anchor of FIG. 1 with an end portion guide;

FIGS. 11A and 11B are diagrammatic perspective-view representations of the clasp portion of the suture anchor of FIG. 1 with a locking mechanism;

FIG. 12 is diagrammatic perspective-view representation of an alternative embodiment of the suture anchor of FIG. 1 with a locking mechanism and locking suture; and

FIG. 13 is diagrammatic perspective-view representation of the suture anchor of FIG. 1 with a cannulated locking screw to facilitate securing the expansion portion in the expanded position.

### DETAILED DESCRIPTION OF EMBODIMENTS

Further features of the present invention are more fully described in the following description of several non-limiting embodiments thereof. This description is included solely for the purposes of exemplifying the present invention to the skilled addressee. It should not be understood as a restriction on the broad summary, disclosure or description of the invention as set out above. In the figures, incorporated to illustrate features of the example embodiment or embodiments, like reference numerals are used to identify like parts throughout.

Referring now to the accompanying drawings, there is shown one embodiment of a suture anchor 10 for securing soft tissue 12 to bone 6. The suture anchor 10 generally comprises an elongate strip 14 having first and second end portions 16 and 18 and a central portion 20 therebetween. The strip 14 is typically manufactured from medical-grade polymer material, but other materials are also possible and within the scope of the present invention.

Each end portion 16 and 18 defines an inner and an outer aperture 22 and 24, respectively, as shown. The central portion 20 defines a central aperture 21 and transverse lines of weakness 26 longitudinally on either side of the central aperture 21, as shown. It is to be appreciated that the lines of weakness 26 generally runs transverse across a length of the strip 14 and is longitudinally, i.e. spaced along a length of the strip 14, on either side of the central aperture 21, as shown.

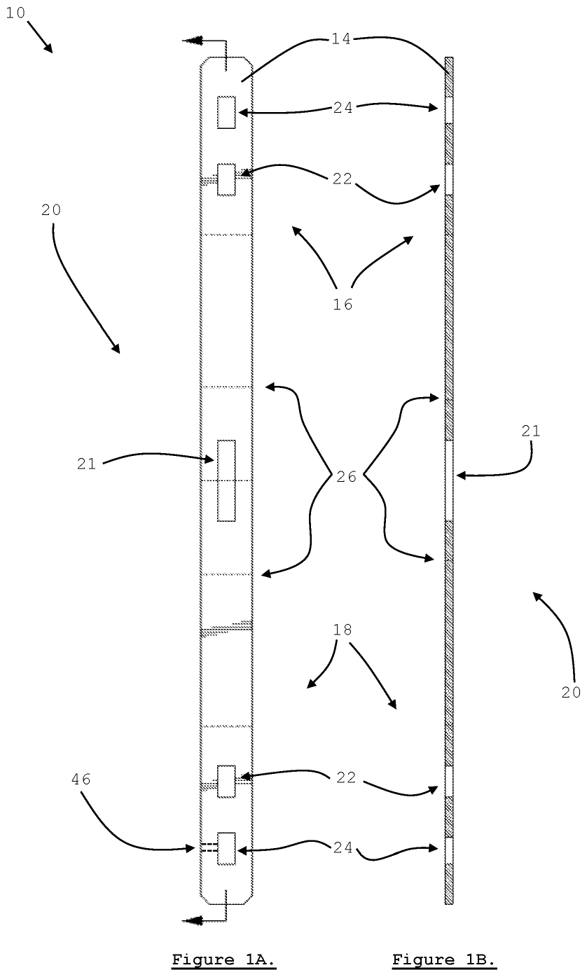

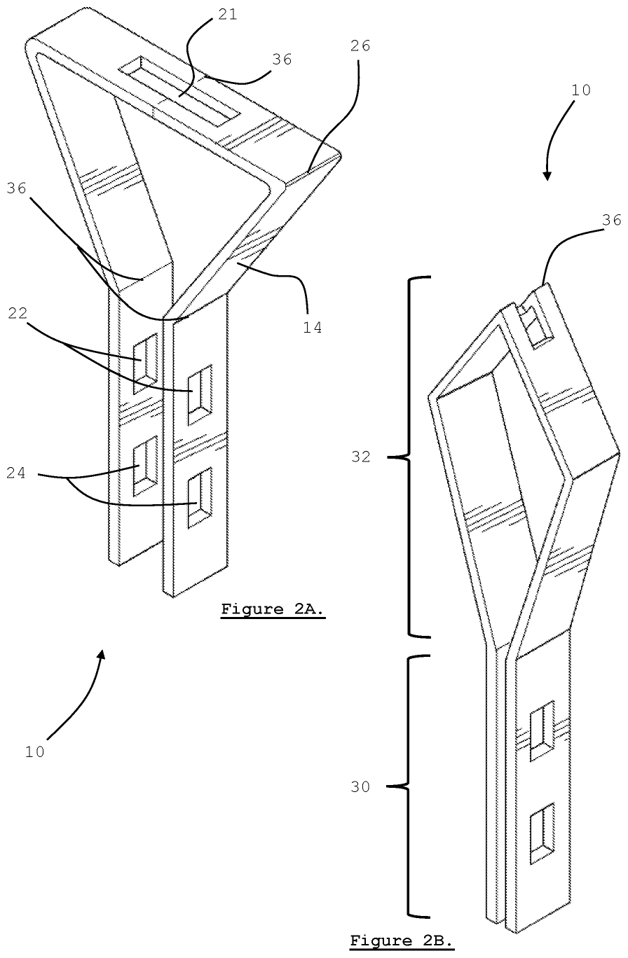

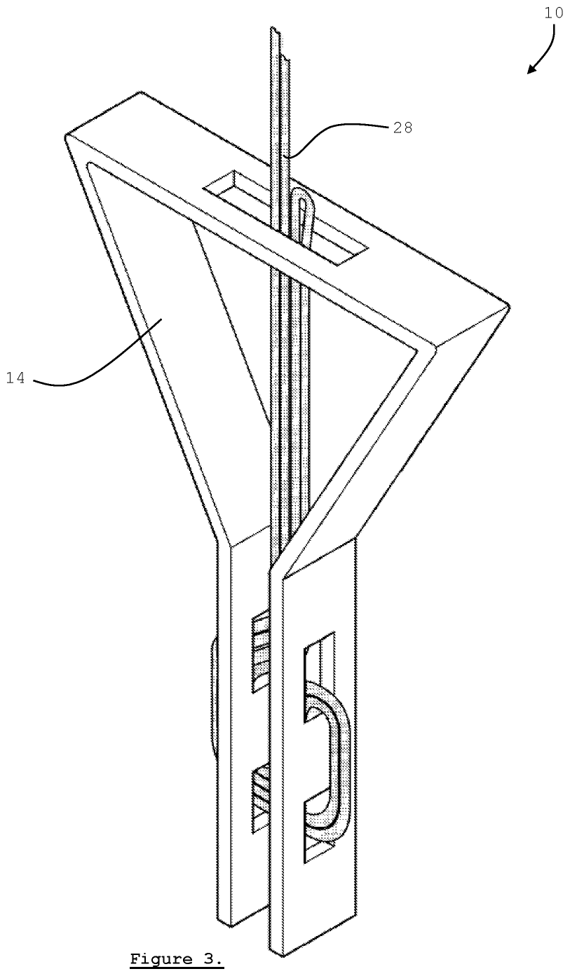

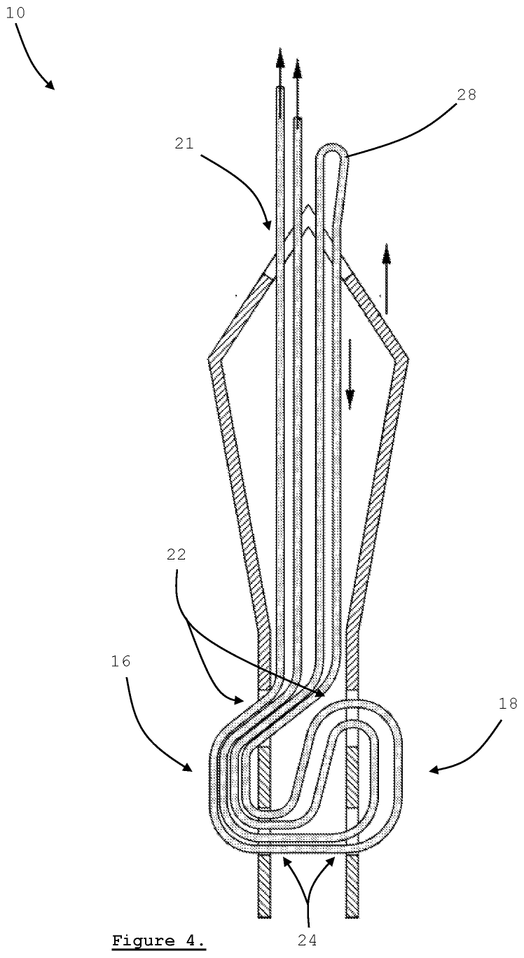

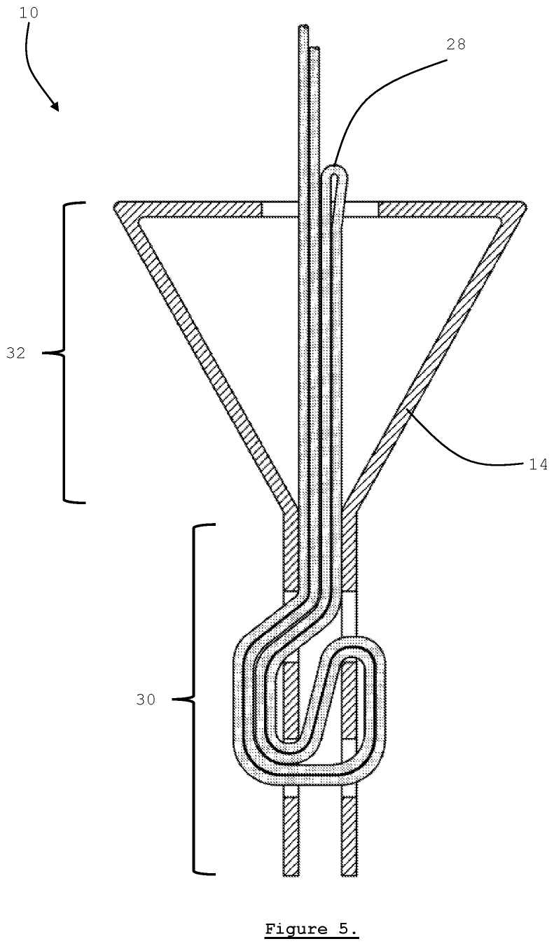

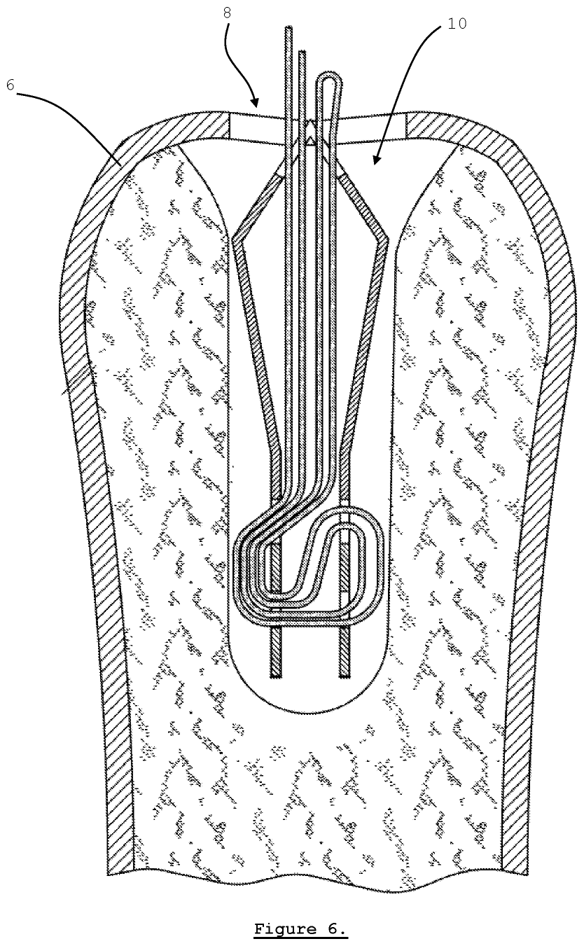

In an embodiment, shown in FIG. 1A, the first and/or second end portions 16, 18 may define one or more opening 46 leading into the inner and/or outer apertures 22, 24 for a suture 28 to operatively pass through, said opening 46 configured to allow suture entry into said inner and/or outer aperture 22, 24 while substantially impeding suture exit therefrom. As indicated in the exemplified embodiment, the opening 46 is configured as a narrow entry point for the suture 46, wherein the suture can be guided into the inner and/or outer aperture 22, 24, with a width of the opening such that exit of the suture is substantially impeded. Other variations hereon are possible and within the scope of the invention, e.g. a barb at the opening 46, decreasing width or a notched opening 46 etc.

In use, a suture 28 is operatively threadable through the strip 14 such that the end portions 16 and 18 fold onto each other, as shown, via the lines of weakness 26 to complementarily define a clasp portion 30, with the central portion 20 defining an expansion portion 32. In this manner, when in use, tensioning of the suture 28 generally urges the expansion portion 32 into an expanded position and clasps said suture 28 to the clasp portion 30.

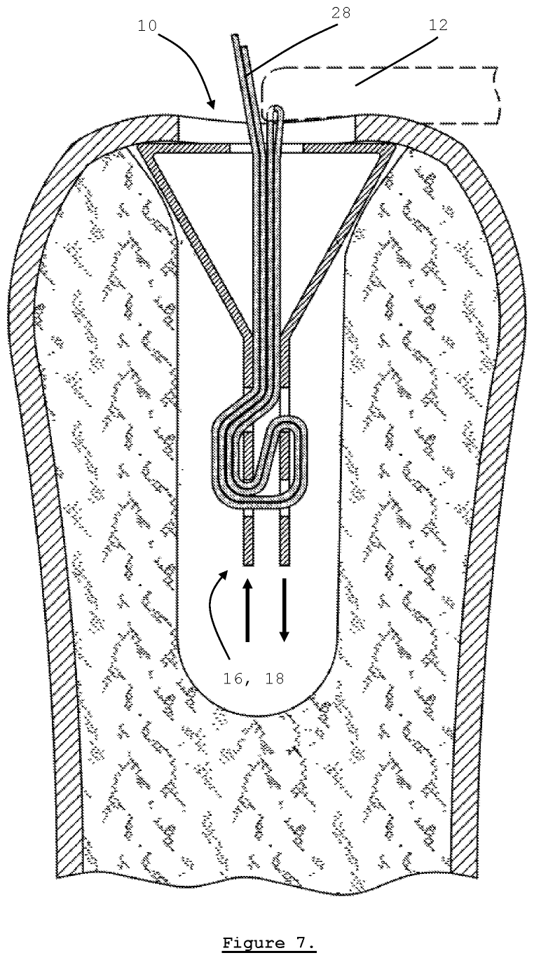

In one example, as shown more clearly in FIG. 7, such tensioning of the suture 28 pulls or draws the end portions 16 and 18 together and may also cause these end portions 16 and 18 to slip relative to each other. In this manner, the suture 28 is generally clasped, captured or pinched between said end portions 16 and 18. As the suture 28 is generally fastened to soft tissue 12, the tension therein is maintained, keeping the strip 14 in the expanded position and maintaining tension throughout the suture 28 which keeps the suture clasped in place.

In the present example, the suture 28 is sequentially threaded through the strip 14 as follows:
i) through the central aperture 21;
ii) via the inner aperture 22 in the first end portion 16;
iii) via the outer aperture 24 in the first end portion 16;
iv) via the outer aperture 24 in the second end portion 18;
v) via the inner aperture 22 in the second end portion 18;
vi) returning to the outer aperture 24 in the first end portion 16;
vii) via the inner aperture 22 in the first end portion 16; and
viii) exiting the strip 14 via the central aperture 20, as shown.

In one embodiment, the clasp portion 30 may include a rough surface texture to facilitate in operatively clasping the suture 28, or the like.

The skilled addressee will appreciate that the strip 14 may include further lines of weakness 36 to facilitate in the expansion portion 32 being urged into the expanded position. In the present embodiment, the strip 14 defines an additional line of weakness 36 transversely across the central aperture 21, as shown, as well as two additional lines of weakness 36 which generally separates the clasp portion 30 from the expansion portion 32.

With reference now to FIG. 8 of the accompanying drawings, the skilled addressee will appreciate that variations on the above-described embodiment are possible and within the scope of the present invention. For example, in another embodiment, the strip 14 of the suture anchor 10 may include an additional aperture 34 to allow for variations on the threading of the suture 28. In other embodiments, the suture 28 may be threaded in alternative manners to that described herein. Such other threading arrangements are included and within the scope of the present invention.

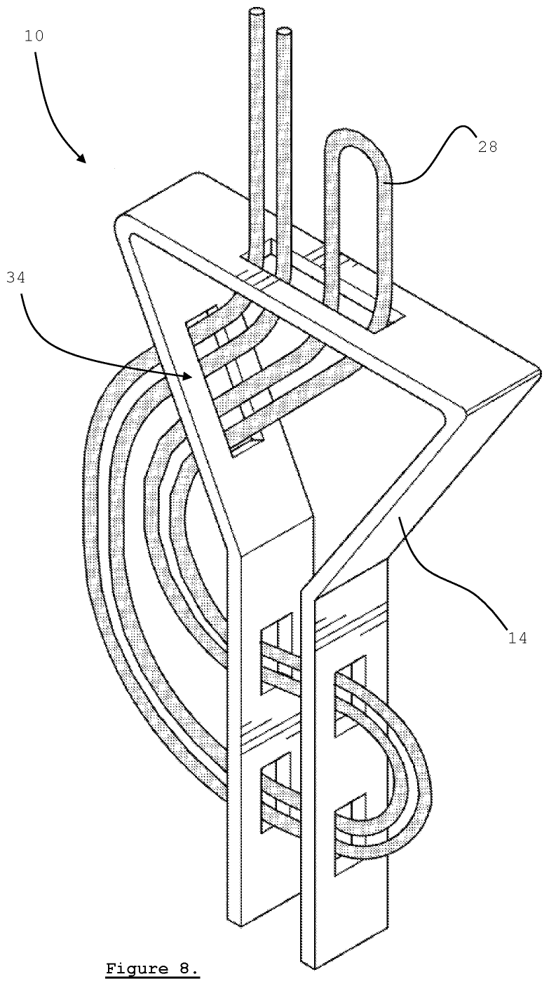

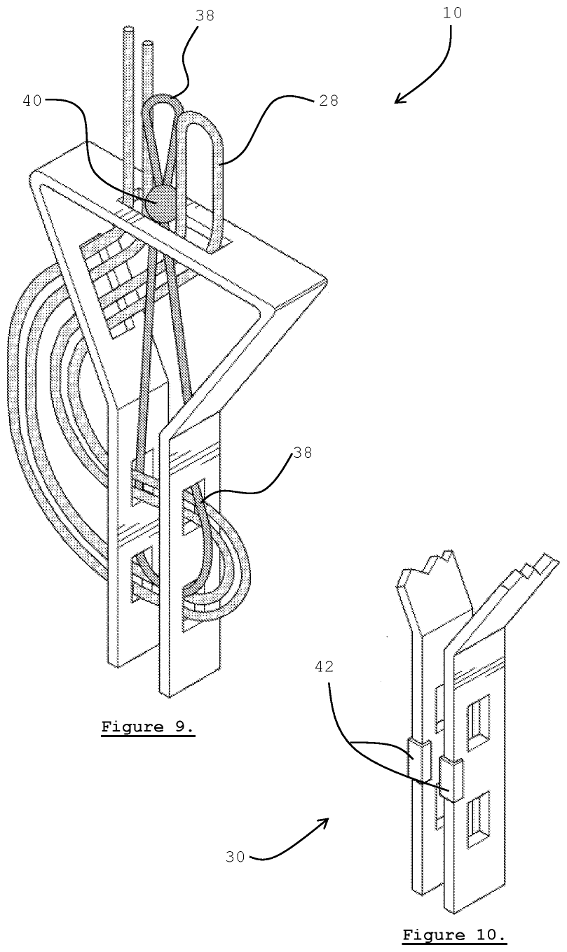

In the embodiment of FIG. 9, the suture anchor 10 includes a locking suture 38 which is operatively threaded through the strip 14 and having a detent 40 which is configured to lock the expansion portion 32 into the expanded position, as shown. In this embodiment, the detent 40 of the locking suture 38 comprises a bulge 40 sized, shaped and dimensioned for interference fit through the central aperture 21 of the strip 14. The skilled addressee will appreciate that the detent may also fit through another aperture of the strip 14, as per requirements. Usefully, the locking suture 38 can further be used as anchor point for attaching tissue, or the like.

Referring to the embodiment shown in FIG. 10, the strip 14 may further include some manner of end portion guide 42 which configured to guide the end portions 16, 18 once folded onto each other, with the end portion guide 42 typically configured to allow sliding of such end portions 16, 18 longitudinally relative to each other, i.e. to allow some slip along their respective lengths to facilitate operation of the clasp portion 30 in securing the suture 28. The skilled addressee will appreciate that the end portion guide 42 may take various forms, as is diagrammatically indicated by reference numeral 42, such as a guiding protrusion, projecting edges, tongue-and-groove arrangements, etc.

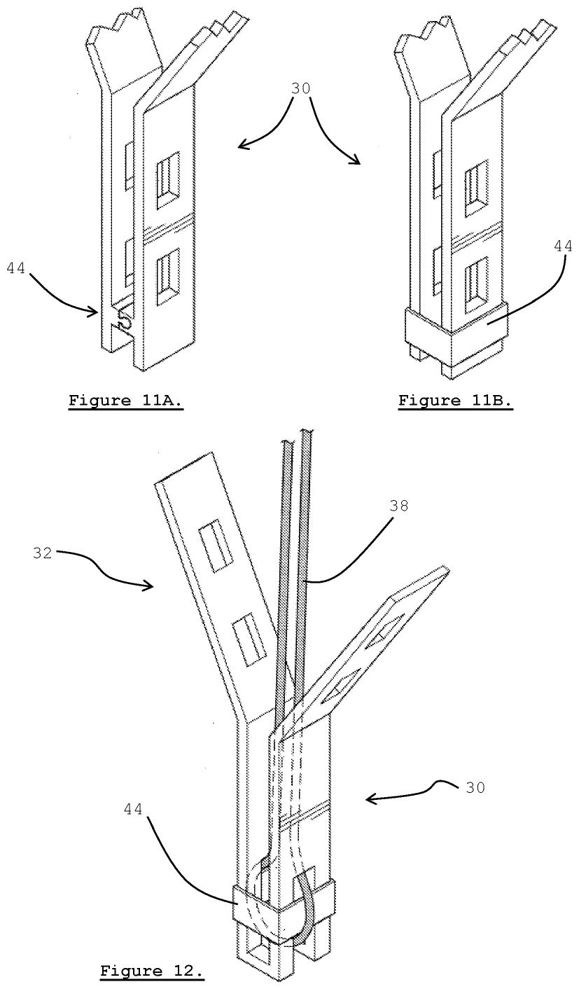

In a further embodiment, exemplified in FIG. 11, the strip 14 includes a locking mechanism 44 which is configured to lock the end portions 16, 18 together once folded onto each other. The locking mechanism 44 may take various forms, depending on requirement, and is generally configured to complementarily engage the end portions 16, 18 to facilitate the clasp portion 30 clasping the suture 28. The locking mechanism 44 may include an interference-fit arrangement, a sliding clasp, etc.

It is to be appreciated that further configurations of the suture anchor 10 are possible and within the scope of the present invention. For example, the embodiment of FIG. 12 shows a version where the strip 14 is configured such that the expansion portion 32 is comprised of the end portions 16, 18 of the strip, with the clasp portion 30 comprised of the central portion 20. This version also includes the locking mechanism 44, as shown, and locking suture 38 may even replace the suture 28, or both may be used, etc. In this version, the clasp portion 30 is inserted into the bone channel 8 first, but the overall operation of the anchor 10 remains generally as described above.

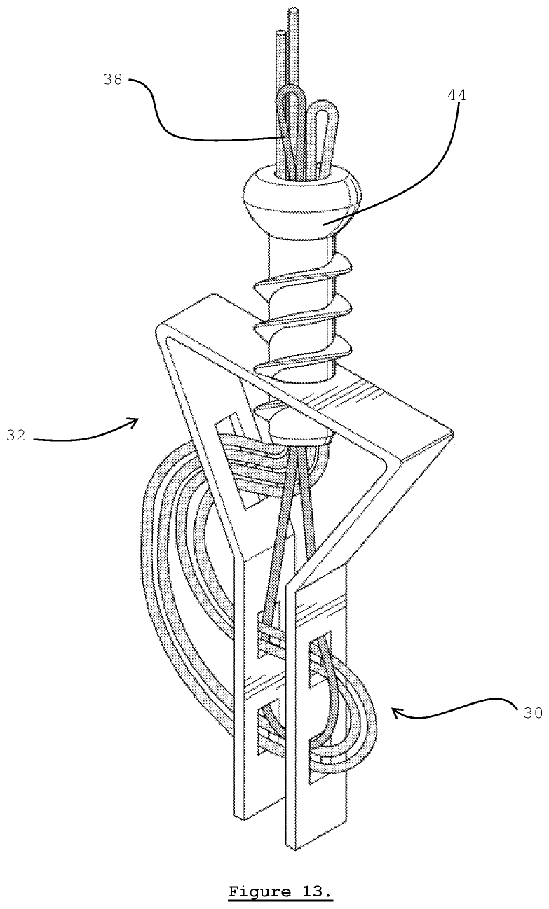

Similarly, a yet further configuration is shown in FIG. 13, where the suture anchor 10 includes the locking mechanism 44 in the form of a cannulated screw through which the suture 28 and/or locking suture 38 (noting that locking suture 38 is optional) may pass, as shown, with the screw 44 operatively securing the expansion portion 32 in the expanded position, such as being screwed into the bone channel 8, optionally through the central portion 20, and/or the like.

The invention further provides for an associated method for securing soft tissue 12 to bone 6. The method generally comprises the steps of providing the suture anchor 10 as described above, threading the suture 28 through the strip 14 such that the end portions 16 and 18 fold onto each other via the lines of weakness 26 to complementarily define the clasp portion 30 and the expansion portion 32, as described, inserting the folded strip 14 clasp portion-first into a bone channel 8, and tensioning the suture 28 to urge the expansion portion 32 into an expanded position to secure the strip 14 inside the bone channel 8 whilst clasping the suture 28 to the clasp portion 30.

Typically, the method includes the step of attaching the suture 28 to the soft tissue 12 to be secured proximate the bone channel 8.

Applicant believes is particularly advantageous that the present invention provides for a simple and elegant suture anchor useable for attaching soft tissue 12 to bone.

Optional embodiments of the present invention may also be said to broadly consist in the parts, elements and features referred to or indicated herein, individually or collectively, in any or all combinations of two or more of the parts, elements or features, and wherein specific integers are mentioned herein which have known equivalents in the art to which the invention relates, such known equivalents are deemed to be incorporated herein as if individually set forth. In the example embodiments, well-known processes, well-known device structures, and well-known technologies are not described in detail, as such will be readily understood by the skilled addressee.

The use of the terms “a”, “an”, “said”, “the”, and/or similar referents in the context of describing various embodiments (especially in the context of the claimed subject matter) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. As used herein, the term “and/or” includes any and all combinations of one or more of the associated listed items. No language in the specification should be construed as indicating any non-claimed subject matter as essential to the practice of the claimed subject matter.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

It is to be appreciated that reference to “one example” or “an example” of the invention, or similar exemplary language (e.g., “such as”) herein, is not made in an exclusive sense. Various substantially and specifically practical and useful exemplary embodiments of the claimed subject matter are described herein, textually and/or graphically, for carrying out the claimed subject matter.

Accordingly, one example may exemplify certain aspects of the invention, whilst other aspects are exemplified in a different example. These examples are intended to assist the skilled person in performing the invention and are not intended to limit the overall scope of the invention in any way unless the context clearly indicates otherwise. Variations (e.g. modifications and/or enhancements) of one or more embodiments described herein might become apparent to those of ordinary skill in the art upon reading this application. The inventor(s) expects skilled artisans to employ such variations as appropriate, and the inventor(s) intends for the claimed subject matter to be practiced other than as specifically described herein.

Any method steps, processes, and operations described herein are not to be construed as necessarily requiring their performance in the particular order discussed or illustrated, unless specifically identified as an order of performance. It is also to be understood that additional or alternative steps may be employed.

1. A suture anchor for securing soft tissue to bone, said suture anchor comprising:
a strip having first and second end portions and a central portion therebetween, each end portion defining an inner and an outer aperture, said central portion defining a central aperture and transverse lines of weakness longitudinally on either side of said central aperture;
wherein a suture is operatively threaded through the strip such that the end portions fold onto each other via the lines of weakness to complementarily define a clasp portion with the central portion defining an expansion portion; and
wherein, in use, tensioning of said suture urges the expansion portion into an expanded position and clasps said suture to the clasp portion.

2. The suture anchor of claim 1, wherein the suture is sequentially threaded through the strip, in use, as follows:
i) through the central aperture;
ii) via the inner aperture in the first end portion;
iii) via the outer aperture in the first end portion;
iv) via the outer aperture in the second end portion;
v) via the inner aperture in the second end portion;
vi) returning to the outer aperture in the first end portion;
vii) via the inner aperture in the first end portion; and
viii) exiting the strip via the central aperture.

3. The suture anchor of claim 1, wherein the strip is manufactured from medical-grade polymer material.

4. The suture anchor of claim 1, wherein the clasp portion defines a rough surface texture to facilitate in operatively clasping the suture.

5. The suture anchor of claim 1, wherein the strip includes further lines of weakness to facilitate in the expansion portion urging into the expanded position.

6. The suture anchor of claim 1, wherein the strip defines an additional line of weakness transversely across the central aperture.

7. The suture anchor of claim 1, wherein the strip defines two additional lines of weakness to separate the clasp portion from the expansion portion.

8. The suture anchor of claim 1, which includes a locking suture operatively threaded through the strip and having a detent configured to lock the expanded portion into the expanded position.

9. The suture anchor of claim 8, wherein the detent of the locking suture comprises a bulge sized, shaped and dimensioned for interference fit through an aperture of the strip.

10. The suture anchor of claim 1, wherein the strip includes an end portion guide configured to guide the end portions once folded onto each other, the end portion guide configured to allow sliding of such end portions longitudinally relative to each other.

11. The suture anchor of claim 1, wherein the strip includes a locking mechanism configured to lock the end portions together once folded onto each other.

12. The suture anchor of claim 11, wherein the locking mechanism is configured to complementarily engage the end portions to facilitate the clasp portion clasping the suture.

13. The suture anchor of either of claim 12, wherein the locking mechanism includes a cannulated screw configured to facilitate in urging and/securing the expansion portion into the expanded position.

14. The suture anchor of claim 1, wherein the first and/or second end portions define one or more opening leading into the inner and/or outer apertures for a suture to operatively pass through, said opening configured to allow suture entry into said inner and/or outer aperture, while substantially impeding suture exit therefrom.

15. A method for securing soft tissue to bone, said method comprising the steps of:
providing a suture anchor comprising a strip having first and second end portions and a central portion therebetween, each end portion defining an inner and an outer aperture, said central portion defining a central aperture and transverse lines of weakness longitudinally on either side of said central aperture;
threading a suture through the strip such that the end portions fold onto each other via the lines of weakness to complementarily define a clasp portion with the central portion defining an expansion portion, said suture attachable to soft tissue;
inserting the folded strip clasp portion-first into a bone channel; and
tensioning the suture to urge the expansion portion into an expanded position to secure said strip inside said bone channel and to clasp said suture to the clasp portion, so that said soft tissue is anchorable to the suture anchor secured within the bone channel.

16. The method of claim 15, which includes the step of attaching the suture to soft tissue to be secured proximate the bone channel.

17. The method of claim 15, wherein the step of threading the suture includes sequentially threading the suture:
i) through the central aperture;
ii) via the inner aperture in the first end portion;
iii) via the outer aperture in the first end portion;
iv) via the outer aperture in the second end portion;
v) via the inner aperture in the second end portion;
vi) returning to the outer aperture in the first end portion;
vii) via the inner aperture in the first end portion; and
viii) exiting the strip via the central aperture.

18. (canceled)
